(19) United States
(12) Patent Application Publication
(10) Pub. No.: US 2013/0211347 A1
(43) Pub. Date: Aug. 15, 2013

(54) STIFFENER FOR USE WITH A DRUG PATCH

(71) Applicants: Giora Arbel, Tel Mond (IL); Yossi Bar-El, Beit Arye (IL)

(72) Inventors: Giora Arbel, Tel Mond (IL); Yossi Bar-El, Beit Arye (IL)

(21) Appl. No.: 13/572,612

(22) Filed: Aug. 10, 2012

Related U.S. Application Data
(63) Continuation of application No. PCT/IL2011/000315, filed on Apr. 14, 2011.
(60) Provisional application No. 61/324,376, filed on Apr. 15, 2010.

(51) Int. Cl. A61K 9/70

(52) U.S. Cl. CPC A61K 9/7084
USPC 604/290; 604/308; 604/307

(57) ABSTRACT

Apparatus and methods are provided including a patch assembly (20) configured to deliver a substance through a subject's skin. A frame (26) surrounds at least in part an open portion of the patch assembly, such that a region of the skin is exposed through the open portion when the frame is placed on the skin. A substance support area (28) is foldably coupled to the frame at a foldable joint (27). A substance delivery area (23) is adapted to be placed in contact with at least a portion of the region of skin, by the substance support area being folded with respect to the frame. A stiffener (29), disposed on the substance support area between the foldable joint and the substance delivery area, is configured to facilitate the placement of the substance delivery area on the portion of the region of skin. Other applications are also described.

### CROSS-REFERENCE TO RELATED APPLICATIONS

This application is being filed under 37 U.S.C. 111 as a continuation application of International Application Number PCT/IL2011/000315, which has an international filing date of Apr. 14, 2011 and which claims priority to the following United States provisional application for patent: Ser. No. 61/324,376 filed on Apr. 15, 2010. This application claims the benefit of the priority date Apr. 15, 2010 under 37 U.S.C. 120 as a continuation of PCT/IL2011/000315 which claims priority as previously stated. The International Application Number PCT/IL2011/000315 is co-pending at the filing of this application and includes at least one common inventor. This application incorporates the above-identified applications by reference in their entirety.

### FIELD OF EMBODIMENTS OF THE INVENTION

Some applications of the present invention relate generally to methods and devices for drug delivery and specifically to methods and devices for transdermal drug delivery.

### BACKGROUND

Adhesive transdermal drug patches deliver a drug across the skin directly into the systemic blood circulation. Typically, the drug is dispersed in the adhesive that attaches the patch to the skin.

### BRIEF SUMMARY

For some applications of the present invention, a patch assembly is used for facilitating delivery of a substance (e.g., a drug) through a subject's skin. For some applications, the patch assembly is generally similar to those described in US 2008/0274166 to Sacks, WO 03/039620 to Sohn, US 2005/0119605 to Sohn, US 2010/0286588 to Bar-El, and/or in WO 09/047,774 to Bar-El, all of which applications are incorporated herein by reference. The patch assembly typically comprises first and second portions. The first portion includes a frame that surrounds an open portion of the patch assembly. The frame is placed on the skin such that a region of the skin is exposed through the open portion. The second portion of the patch assembly includes a pad that includes the substance that is to be delivered through the subject's skin. The substance is delivered through the subject's skin by the pad being brought into contact, through the open portion, with the region of the skin that is exposed through the open portion. The first and second portions of the patch assembly are foldably coupled to each other at a foldable joint. The pad is brought into contact with the region of skin that is exposed through the open portion by the second portion being folded with respect to the first portion, at the foldable joint. For some applications, a stiffener is disposed adjacent to the foldable joint, and stiffens the patch in the region adjacent to the joint. Typically, this facilitates the folding of the second portion of the patch assembly with respect to the first portion.

For some applications, the pad has a skin-facing non-absorbent layer. For example, the non-absorbent layer may include polyethylene, low density polyethylene, high density polyethylene, and/or ethylene vinyl acetate. The substance, in dried form is disposed on the non-absorbent layer. For example, the substance may be lyophilized, or dried using convection, and then printed onto the non-absorbent layer. The non-absorbent layer of the pad is placed on the skin in order to deliver the substance to the subject via the skin. For some applications, the substance is wetted, upon the patch being placed into contact with the subject's skin. For example, before the pad has been placed on the subject's skin, the subject's skin may have been ablated using the techniques described in U.S. Pat. No. 6,148,232 to Avrahami, which is incorporated herein by reference. The non-absorbent layer prevents resorption of the substance into the pad after the substance has been wetted.

For some applications, the frame of the patch assembly prevents the substance from spreading across the subject's skin beyond the region of the skin, by sealing the region of the skin, as described in further detail hereinbelow.

There is therefore provided, in accordance with some applications of the present invention, apparatus for facilitating delivery of a substance through skin of a subject, including:

a patch assembly configured to deliver the substance through the skin by being placed on the subject's skin, the patch assembly including:

a frame that surrounds at least in part an open portion of the patch assembly, such that a region of the skin is exposed through the open portion when the frame is placed on the skin;

a substance support area that is foldably coupled to the frame at a foldable joint; and

a substance delivery area disposed on the substance support area, and including the substance, the substance delivery area being adapted to be placed in contact, through the open portion, with at least a portion of the region of the skin, by the substance support area being folded with respect to the frame; and a stiffener disposed on a region of the substance support area between the foldable joint and the substance delivery area, configured to facilitate the placement of the substance delivery area on the portion of the region of the skin.

For some applications, the stiffener is configured to reduce folding of the region of the substance support area between the foldable joint and the substance delivery area.

For some applications, the stiffener is configured to prevent the substance delivery area from being placed on the frame, when the substance support area is folded with respect to the frame.

For some applications, the stiffener has a thickness of 50 μm to 200 μm.

For some applications, the stiffener includes a stiffening material selected from the group consisting of: a polyester, polyethylene terephthalate, polyethylene, an ethylene vinyl acetate, and a polymer film.

For some applications, the substance delivery area defines a skin-facing non-absorbent surface.

For some applications, the substance includes a dried substance configured to be delivered through the skin, by being disposed on the non-absorbent surface and by the non-absorbent surface being placed on a wetted portion of the skin, the non-absorbent surface being configured to prevent resorption of the substance into the substance delivery area after the substance has been wetted.

For some applications, the apparatus further includes a skin preparation device including one or more electrodes, and configured to wet the substance by facilitating release of fluid from the skin by ablating the subject's skin.

For some applications, the non-absorbent surface has a surface tension of more than 40 dynes/cm.

For some applications, the substance delivery area is configured to prevent the substance from spreading across the subject's skin beyond the region of the skin, by generating protrusions in the skin at outer edges of the region, by the substance delivery area applying pressure to the portion of the region of the skin.

For some applications, the substance delivery area has a thickness of 0.5 mm to 1.5 mm.

For some applications, the substance delivery area is configured to apply the pressure to the skin underneath the substance delivery area, by the substance support area being stretched when the substance delivery area is brought into contact with the portion of the region of the skin.

For some applications, the patch assembly further includes a first adhesive layer disposed on the substance support area, and subsequent to the substance delivery area being brought into contact with the portion of the region of the skin, the first adhesive layer is configured to adhere the substance support area to the frame.

For some applications, the patch assembly further includes a second adhesive layer disposed on the frame and configured to adhere the frame to the subject's skin.

For some applications, a tackiness of the second adhesive layer is greater than a tackiness of the first adhesive layer.

For some applications, the first adhesive layer is composed of a substance that is the same as a substance of which the second adhesive layer is composed, and the second adhesive layer is thicker than the first adhesive layer.

For some applications, the second adhesive layer includes a substance that the first adhesive layer does not include.

There is further provided, in accordance with some applications of the present invention, a method for delivering a substance through skin of a subject, including: placing on the skin a frame of an assembly that surrounds at least in part an open portion of the assembly, such that a region of the skin is exposed through the open portion when the frame is placed on the skin; bringing a pad into contact, through the open portion, with at least a portion of the region of the skin, the pad having the substance disposed thereon; and preventing the substance from spreading across the subject's skin beyond the region of the skin, by generating protrusions in the skin at outer edges of the region, by applying pressure, with the pad, to the portion of the region of the subject's skin.

For some applications, applying the pressure with the pad includes stretching a substance support area on which the pad is disposed, while the pad is brought into contact with the portion of the region of the skin.

For some applications, preventing the substance from spreading across the subject's skin beyond the region of the skin includes adhering a substance support area to the frame, the pad being disposed on the substance support area.

There is additionally provided, in accordance with some applications of the present invention, apparatus for use with skin of a subject, including:

a dried substance configured to be delivered through the skin; and

a patch assembly configured to be placed on the skin, the patch assembly including:

a frame that surrounds at least in part an open portion of the patch assembly, such that a region of the skin is exposed through the open portion when the frame is placed on the skin; and

a pad having a skin-facing non-absorbent layer, the dried substance being disposed on the skin-facing non-absorbent layer, and the skin-facing non-absorbent layer being configured to:

deliver the dried substance through the skin by being brought into contact, through the open portion, with at least a portion of the region of the skin, the portion of the region of the skin having been wetted, and

prevent resorption of the substance into the pad after the substance has been wetted,

the pad being configured to prevent the substance from spreading across the subject's skin beyond the region of the skin, by generating protrusions in the skin at outer edges of the region, by the pad applying pressure to the portion of the region of the skin.

There is still further provided, in accordance with some applications of the present invention, apparatus for use with skin of a subject, including:

a pad having a skin-facing non-absorbent surface; and

a dried substance configured to be delivered through the skin, by being disposed on the non-absorbent surface of the pad and by the non-absorbent surface of the pad being placed on a wetted portion of the skin, the non-absorbent surface being configured to prevent resorption of the substance into the pad after the substance has been wetted.

For some applications, the apparatus further includes a skin preparation device including one or more electrodes, and configured to wet the substance by facilitating release of fluid from the skin by ablating the subject's skin.

For some applications, the non-absorbent surface has a surface tension of more than 40 dynes/cm.

There is additionally provided, in accordance with some applications of the present invention, a method for use with skin of a subject, including:

providing a pad having a skin-facing non-absorbent surface, a dried form of a substance being disposed on the surface;

causing release of fluid from the skin by ablating the skin; and

delivering the substance through the skin, by wetting the substance, by placing the skin-facing non-absorbent surface of the pad on the ablated skin, the non-absorbent surface preventing resorption of the substance into the pad after the substance has been wetted.

There is further provided, in accordance with some applications of the present invention, apparatus for facilitating delivery of a substance through skin of a subject, including:

a patch assembly configured to deliver the substance through the skin by being placed on the subject's skin, the patch assembly including:

a frame that surrounds at least in part an open portion of the patch assembly, such that a region of the skin is exposed through the open portion when the frame is placed on the skin; and

a pad including the substance, the pad being adapted to be brought into contact, through the open portion, with at least a portion of the region of the skin, the pad being configured to prevent the substance from spreading across the subject's skin beyond the region of the skin, by generating protrusions in the skin at outer edges of the region, by the pad applying pressure to the portion of the region of the skin.

For some applications, the pad has a thickness of 0.5 mm to 1.5 mm.

For some applications, the patch assembly further includes a substance support area, the pad is disposed on the substance support area, and the pad is configured to apply the pressure to the skin underneath the pad, by the substance support area being stretched when the pad is brought into contact with the portion of the region of the skin.

For some applications, the patch assembly further includes a substance support area and a first adhesive layer, the pad is disposed on the substance support area, and subsequent to the pad being brought into contact with the portion of the region of the skin, the first adhesive layer is configured to adhere the substance support area to the frame.

For some applications, the patch assembly further includes a second adhesive layer configured to adhere the frame to the subject's skin.

For some applications, a tackiness of the second adhesive layer is greater than a tackiness of the first adhesive layer.

For some applications, the first adhesive layer is composed of a substance that is the same as a substance of which the second adhesive layer is composed, and the second adhesive layer is thicker than the first adhesive layer.

For some applications, the second adhesive layer includes a substance that the first adhesive layer does not include.

The present invention will be more fully understood from the following detailed description of applications thereof, taken together with the drawings, in which:

### BRIEF DESCRIPTION OF THE SEVERAL VIEWS OF THE DRAWING

FIGS. 1 and 2 are schematic illustrations of a patch assembly, in accordance with some applications of the present invention;

FIG. 3 is a schematic illustration of a support structure that includes a stiffener, in accordance with some applications of the present invention;

FIGS. 4A-B are schematic illustrations of the patch assembly being coupled to the skin preparation device, and of a liner being removed from the patch assembly, in accordance with some applications of the present invention;

FIG. 5 is a schematic illustration of the skin preparation device being applied to a subject's skin, in accordance with some applications of the present invention;

FIGS. 6A and 6B are schematic illustrations of a drug delivery area of a patch assembly being coupled to the subject's skin, in accordance with some applications of the present invention; and

FIG. 7 is a schematic illustration of a substance that has been applied to a subject's skin, in accordance with some applications of the present invention.

### DETAILED DESCRIPTION OF EMBODIMENTS

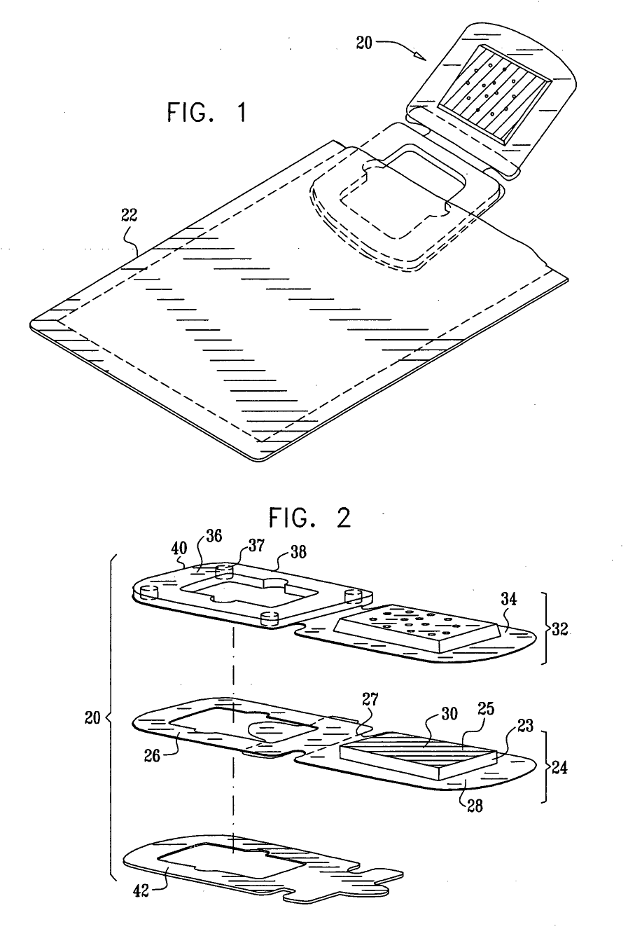

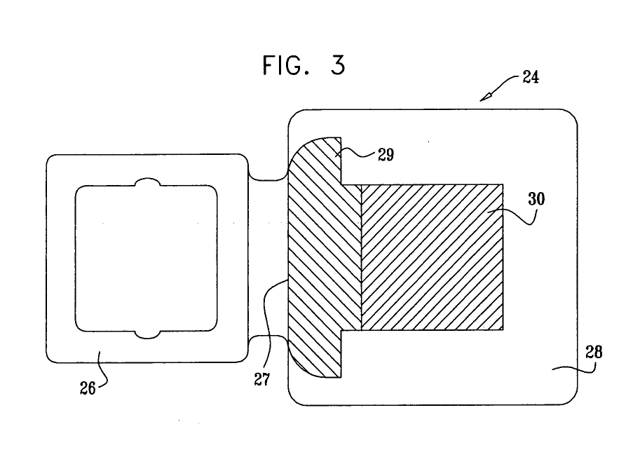

Reference is now made to FIGS. 1 and 2, which are schematic illustrations of a patch assembly 20, in accordance with some applications of the present invention. The patch is typically stored inside packaging 22. Except as described hereinbelow, patch assembly 20 is generally similar to those described in US 2008/0274166 to Sacks, WO 03/039620 to Sohn, US 2005/0119605 to Sohn, US 2010/0286588 to Bar-El, and/or WO 09/047,774 to Bar-El, all of which applications are incorporated herein by reference. Patch 20 is configured to deliver a substance 25, typically a drug, through the skin of a subject.

Patch assembly 20 comprises a support structure 24, which is shaped to define a window area 26 (i.e., a frame) and a substance support area 28 (e.g., a drug support area). The window area and the drug support area are foldably coupled to each other along fold 27. The drug support area supports a substance delivery area 23 (e.g., a drug delivery area). For example, drug delivery area 23 may comprise a medicated pad that includes substance 25, and/or a substance 25 directly adhered to drug support area 28. For some applications, drug delivery area 23 is a pad that has a non-absorbent skin-facing surface 30, as described in further detail hereinbelow. Typically, the pad has a thickness that is greater than 0.5 mm, and/or that is less than 1.5 mm. For example, the thickness of the pad may be between 0.5 mm and 1.5 mm.

Patch assembly 20 comprises a top liner 32, which is shaped to define a liner window area 36 and a protective area 34. Patch assembly 20 further comprises a bottom liner 42 which is configured to be removably coupled to a lower surface of window area 26. For some applications, magnets 37 are disposed inside the liner window area, for coupling the patch assembly to a skin preparation device, as described hereinbelow.

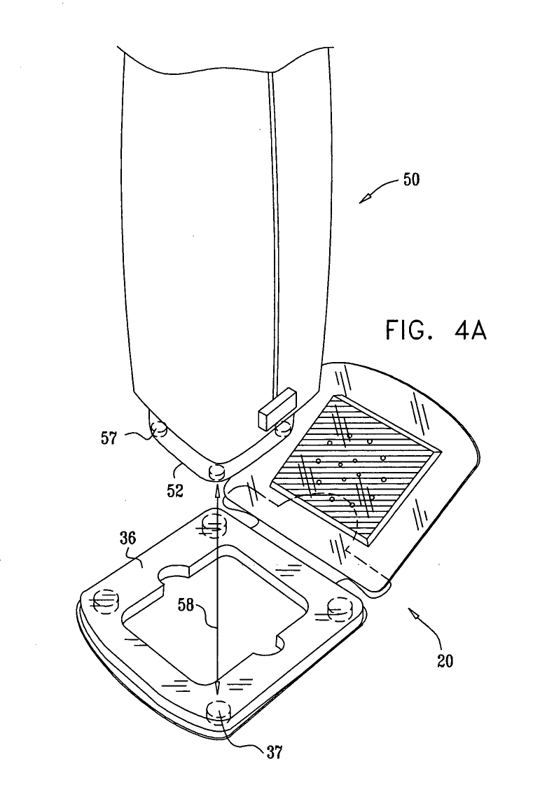

Reference is now made to FIG. 3, which is a schematic illustration of support structure 24, in accordance with some applications of the present invention. For some applications, a stiffener 29 (for example, a stiffening material such as a polyester, polyethylene terephthalate, polyethylene, and/or an ethylene vinyl acetate and/or another polymer film configured to provide stiffening properties) is disposed adjacent to fold 27. Typically, this facilitates the folding of drug support area 28 with respect to window area 26, during use of patch assembly 20, as described in further detail hereinbelow. Further typically, the stiffener is not disposed on other portions of the patch assembly. For some applications, the thickness of the stiffener is more than 50 μη and/or less than 200 μπ [symbol] , e.g., between 50 μπ [symbol] and 200 μπ [symbol] . For example, the thickness of the stiffener may be more than 75 μπ [symbol] and/or less than 125 μη [symbol] , e.g., between 75 μπ [symbol] and 125 μπ [symbol] .

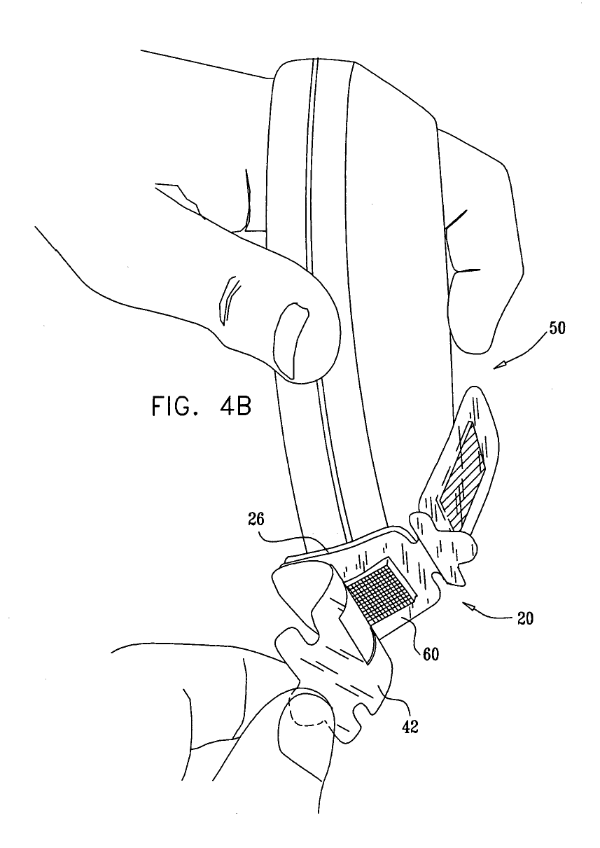

Reference is now made to FIG. 4A, which is a schematic illustration of patch assembly 20 being coupled to a skin preparation device 50, in accordance with some applications of the present invention. For some applications, patch assembly 20 is coupled to skin preparation device 50, and the skin preparation device adheres the patch assembly to the subject's skin, for example, as described in US 2010/0286588 to Bar-El, and/or in PCT Publication WO 09/047,774 to Bar-El, both of which applications are incorporated herein by reference. For example, the skin preparation device may be a device that ablates the stratum corneum of the subject's skin, as described in U.S. Pat. No. 6,148,232 to Avrahami, which is incorporated herein by reference. For some applications, magnets 37 are disposed inside liner window area 36 of the patch assembly. The magnets couple the patch assembly to magnetic material or magnets 57 of skin preparation device 50. Arrow 58 shows the attractive magnetic force between magnets 37 of the patch assembly and magnets 57 of the skin preparation device. The magnetic coupling of the patch assembly to the skin preparation device is typically in accordance with the techniques described in US 2010/0286588 to Bar-El, and/or in PCT Publication WO 09/047,774 to Bar-El, both of which applications are incorporated herein by reference.

Reference is now made to FIG. 4B, which is a schematic illustration of bottom liner 42 being removed from a bottom surface 60 of window area 26 of patch assembly 20, in accordance with some applications of the present invention. In applications of the invention in which skin preparation device 50 is used to adhere the patch assembly to the subject's skin, the bottom liner is typically removed from the window area of the patch assembly subsequent to the patch assembly having been coupled to the skin preparation device, as shown in FIG. 4B.

Alternatively, the patch assembly is not coupled to a skin preparation device.

For example, the subject's skin may first be prepared by ablating the subject's skin (or by preparing the skin using a different method), and subsequently, the subject removes the bottom liner from the window area of the patch assembly, and adheres the patch assembly to his/her skin. For example, the patch assembly may be used in accordance with techniques described in US 2008/0274166 to Sacks, WO 03/039620 to Sohn, and/or US 2005/0119605 to Sohn, all of which applications are incorporated herein by reference.

Typically, bottom surface 60 of window area 26 of patch assembly 20 includes an adhesive layer, and surface 60 is configured to be adhered to the subject's skin via the adhesive layer. For some applications, properties of the adhesive that is used on bottom surface 60 (for sticking the window area to the subject's skin) are different from properties of the adhesive that is used on other portions of the patch (for sticking portions of the patch to each other).

For example, a layer of adhesive may be used on bottom surface 60 that is thicker than layers of adhesive that are used on other portions of the patch. In an application, a layer of adhesive used on bottom surface 60 may be 1.25-2.5 times thicker than layers of adhesive that are used on other portions of the patch. For some applications, a layer of adhesive having a thickness t1 of 0.1-0.2 mm is used on surface 60, and a layer of adhesive having a thickness t2 of 0.06-0.12 mm is used on other portions of the patch, where t1 is greater than t2. Alternatively or additionally, a different adhesive is used on bottom surface 60 from the adhesive that is used on the other portions of the patch. The purpose of the adhesive on surface 60 and on the other surfaces is described in further detail hereinbelow.

For some applications, window area 26 is coupled to, or comprises, a rigid rim configured to dissipate evenly a force applied thereto. By evenly dissipating the force, the rigid rim adheres bottom surface 60 evenly to the subject's skin.

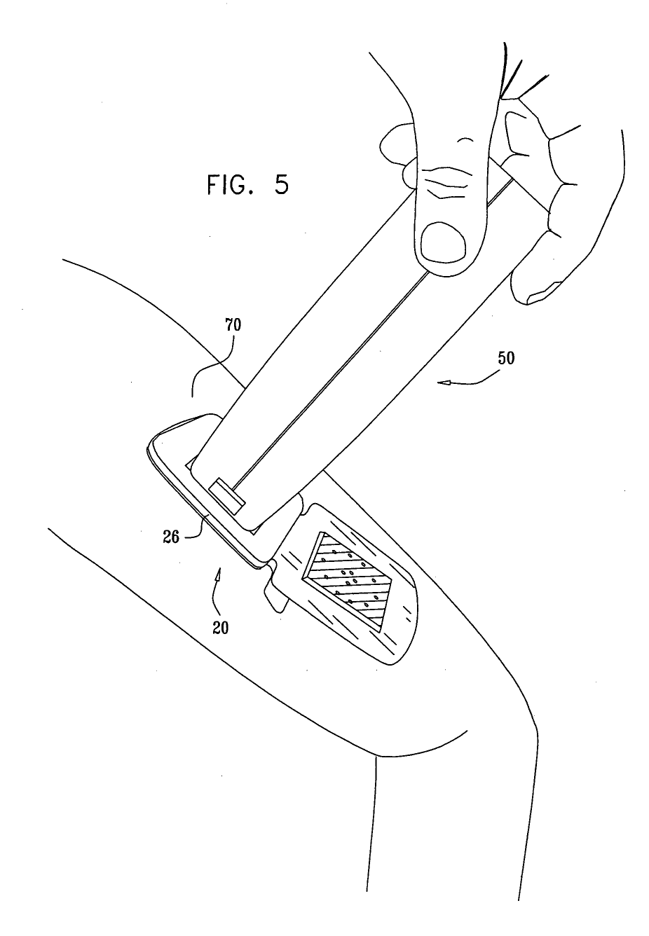

Reference is now made to FIG. 5, which is a schematic illustration of skin preparation device 50 being applied to the subject's skin 70, in accordance with some applications of the present invention. For some applications, the skin preparation device prepares the skin for delivery of a drug from patch assembly 20, by ablating the skin, the skin preparation device comprising ablating electrodes. Typically, bottom surface 60 of window area 26 of patch assembly 20 is adhesive and the skin preparation device is configured to adhere the patch assembly to the skin of the subject.

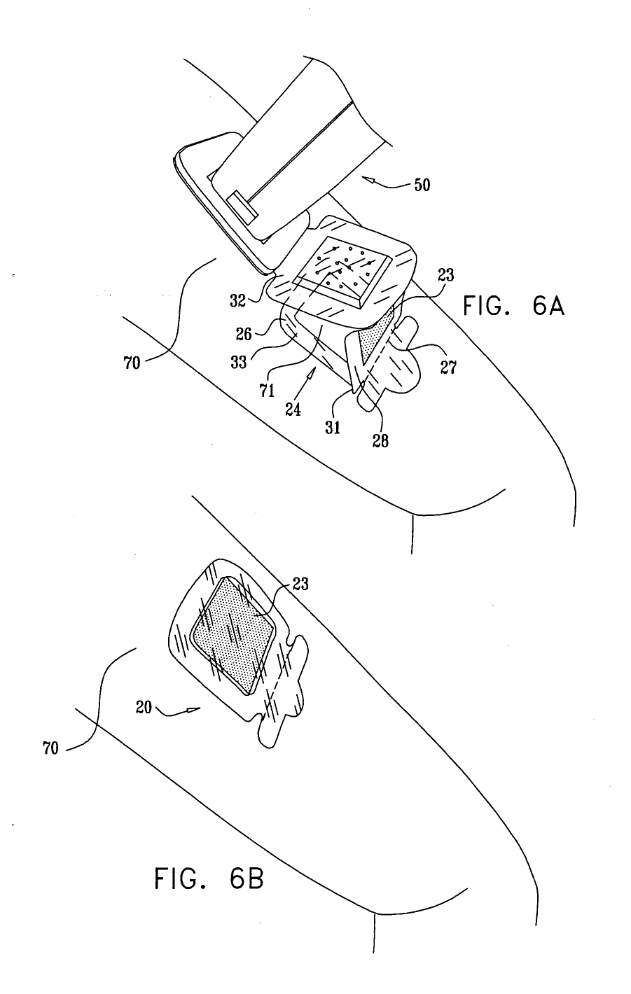

Reference is now made to FIGS. 6A and 6B, which are schematic illustrations of drug delivery area 23 of patch assembly 20 being coupled to a subject's skin 70, in accordance with some applications of the present invention. Skin preparation device 50 has already treated the subject's skin and is being removed from the subject's skin, as shown in FIG. 6A. Window area 26 of the patch assembly remains adhered to the subject's skin leaving a region 71 of skin exposed through the window area.

Typically, as shown in FIG. 6A, as top liner 32 is removed from support structure 24, drug support area 28 is folded over fold 27. Drug delivery area 23 becomes coupled to the subject's skin 70, as shown in FIG. 6B. Alternatively or additionally, drug support area 28 is manually folded over fold 27, and/or drug delivery area 23 is manually coupled to the subject's skin. For some applications, in the absence of stiffener 29 (shown in FIG. 3), the region of drug support area 28 that is between drug delivery area 23 and fold 27 (i.e., the region of the drug support area on top of which the stiffener is disposed) may, in some cases, fold or crease, when the drug delivery area is folded over fold 27. For some applications, the folding of the aforementioned region causes at least a portion of the drug delivery area not to be placed on region 71 of the subject's skin, but rather to be unintentionally placed on the frame of window area 26 of the patch assembly. Typically, stiffener 29 reduces folding of the region of drug support area 28 that is between drug delivery area 23 and fold 27. Thus, stiffener 29 facilitates the coupling of drug delivery area 23 to region 71 of the subject's skin that protrudes through window area 26, by preventing a portion of the drug delivery area from being placed on window area 26.

For some applications, top liner 32 of the patch assembly is coupled to the skin preparation device and is automatically removed from support structure 24 of the patch assembly when the skin preparation device is removed from the skin. For example, top liner 32 of the patch assembly may be magnetically coupled to the skin preparation device, and may be automatically removed from the support structure when the device is removed from the subject's skin, as described in US 2010/0286588 to Bar-El, and/or in PCT Publication WO 09/047,774 to Bar-El, both of which applications are incorporated herein by reference. Typically, in such applications, as a consequence of the removal of the skin preparation device from the skin, all of the magnets of the patch assembly are removed from the skin. Typically, all of the magnets of the patch assembly are removed from the skin because the magnets are disposed in top liner 32 of the patch, assembly, and the top liner is removed from the skin. As a consequence of the removal of the skin preparation device from the skin, only support structure 24 remains on the skin.

For some applications, top liner 32 of the patch assembly is not coupled to the skin preparation device, and is removed from support structure 24 of the patch assembly by the subject, for example, as described in US 2008/0274166 to Sacks, which is incorporated herein by reference.

For some applications, adhesive layers are applied to surface 60 and to other surfaces of the patch. For example, surface 31 of drug support area 28 may have an adhesive layer for adhering surface 31 to a surface 33 of window area 26, during the step shown in FIG. 6A. Surface 60 typically has higher tackiness (i.e., adhesiveness) than the other portions to which adhesive is applied. Typically, the adhesive used to adhere surface 60 to the skin has a level of tackiness that is such that in the transition from FIG. 5 to FIG. 6A (i.e., when top liner 32 of the patch assembly is removed from support structure 24), surface 60 is not removed from the skin. The adhesive for adhering surface 31 to surface 33, for example, is typically not required to have as high a level a tackiness as the adhesive that is used on surface 60. For some applications, the adhesive that is used on surface 60 has a tackiness of 2.5 N/cm to 5 N/cm, e.g., about 4 N/cm. For example, the tackiness may be greater than 2.5 N/cm, and/or less than 5 N/cm.

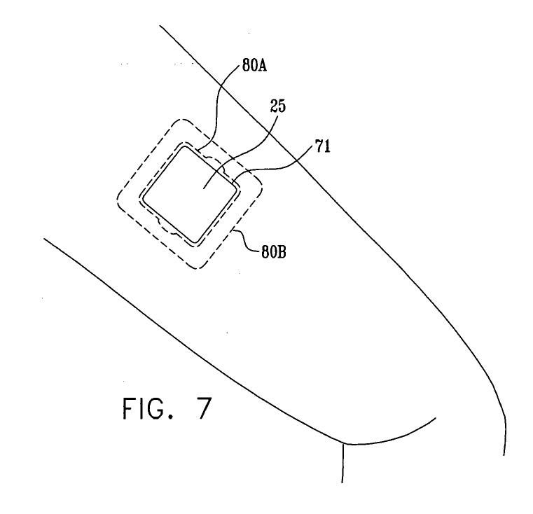

Reference is now made to FIG. 7, which shows substance 25 on region 71 of the subject's skin. For some applications, drug delivery area 23 (e.g., a pad as shown in FIG. 2) is configured to apply pressure to the subject's skin such that the substance is prevented from spreading across the subject's skin beyond region 71. Typically, as shown in FIG. 2, drug delivery area 23 protrudes significantly (for example, 0.5-1.5 mm, e.g., 0.8-1 mm) from drug support area 28, and, although flexible, is sufficiently inflexible such that it can leave a raised mark in the skin surrounding drug delivery area 23, if drug delivery area 23 is pushed firmly and maintained in contact with the skin. For some applications, by applying pressure to the subject's skin that is maintained due to the stretching of drug support area 28 during placement of drug delivery area 23 on the skin, the drug delivery area causes inner protrusion 80A. The inner protrusion is generated between the inner edges of the window area and the outer edges of the region of skin, with which the drug delivery area is brought into contact.

In addition to inner protrusion 80A (which are generated by substance delivery area 23), window area 26 typically generates outer protrusion 80B in the subject's skin by applying pressure to the skin. Inner and outer protrusions 80A and 80B are caused, respectively, by drug delivery area 23 and by window area 26, in a similar manner to that in which socks apply pressure to a wearer's legs, such that marks are formed on the wearer's legs. Inner protrusion 80A is such that the substance is prevented from spreading across the subject's skin beyond region 71, by the inner protrusion sealing the region of the skin. Alternatively, the protrusion reduces, but does not necessarily prevent the substance from spreading across the user's skin.

Typically (although not necessarily) in combination with the above-described features that produce protrusions, skin-facing surface 30 of drug delivery area 23 (shown in FIG. 2, for example) is non-absorbent. Substance 25 is coupled to drug delivery area 23 in dry form, for example, by the substance being printed onto the non-absorbent skin-contacting surface. For some applications, moisture of the subject's body comes in contact with the dry substance stored in patch assembly 20, and dissolves the substance to form a saturated solution or suspension. For example, the moisture may be moisture of the skin of the subject. For some applications, patch assembly 20 is used in conjunction with a skin preparation device (such as device 50, described hereinabove) that forms microchannels through the skin, or otherwise ablates or pre-treats the skin. For such applications, the moisture may be extracellular fluid of the subject released by the body through the microchannels or other openings in the skin. The skin-facing surface of drug delivery area 23 brings the substance into contact with region 71 of skin, thereby causing the substance to become moistened. The non-absorbent nature of the skin-facing surface prevents the drug from being resorbed into drug delivery area 23, thereby causing the concentration of the drug that is absorbed into region 71 of skin to be greater than if skin-facing surface 30 was absorbent.

The inventors have found that the non-absorbent skin-contacting surface, in combination with generation of protrusions as described hereinabove using a thick, drug support area 28, facilitates maintenance of the drug in liquid form at the site where the skin has been treated, whereby passage of the drug through the treated area of the skin is increased.

Typically, surface 30 has a surface tension of more than 40 dynes/cm, in order to facilitate attachment of substance 25 to the surface. For some applications, surface 30 has a surface tension of 40-60 dynes/cm. For some applications, surface 30 is made of polyethylene, and corona treatment is applied to the polyethylene, in order to increase the surface tension thereof.

Although some applications of the present invention are described herein with respect to delivery of a substance into the skin, the scope of the present invention includes using techniques described herein for analyte extraction, mutatis mutandis, or for other purposes.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description

What is claimed is:

1. An apparatus for facilitating delivery of a substance through skin of a subject, comprising:
a patch assembly configured to deliver the substance through the skin by being placed on the subject's skin, the patch assembly comprising:
a frame that surrounds at least in part an open portion of the patch assembly, such that a region of the skin is exposed through the open portion when the frame is placed on the skin;
a substance support area that is foldably coupled to the frame at a foldable joint; and
a substance delivery area disposed on the substance support area, and comprising the substance, the substance delivery area being adapted to be placed in contact, through the open portion, with at least a portion of the region of the skin, by the substance support area being folded with respect to the frame; and
a stiffener disposed on a region of the substance support area between the foldable joint and the substance delivery area, configured to facilitate the placement of the substance delivery area on the portion of the region of the skin.

2. The apparatus according to claim 1, wherein the stiffener is configured to reduce folding of the region of the substance support area between the foldable joint and the substance delivery area.

3. The apparatus according to claim 1, wherein the stiffener is configured to prevent the substance delivery area from being placed on the frame, when the substance support area is folded with respect to the frame.

4. The apparatus according to claim 1, wherein the stiffener has a thickness of 50 μm to 200 μm.

5. The apparatus according to claim 1, wherein the stiffener comprises a stiffening material selected from the group consisting of: a polyester, polyethylene terephthalate, polyethylene, an ethylene vinyl acetate, and a polymer film.

6. The apparatus according to claim 5, wherein the substance delivery area defines a skin-facing non-absorbent surface.

7. The apparatus according to claim 6, wherein the substance comprises a dried substance configured to be delivered through the skin, by being disposed on the non-absorbent surface and by the non-absorbent surface being placed on a wetted portion of the skin, the non-absorbent surface being configured to prevent resorption of the substance into the substance delivery area after the substance has been wetted.

8. The apparatus according to claim 7, further comprising a skin preparation device comprising one or more electrodes, and configured to wet the substance by facilitating release of fluid from the skin by ablating the subject's skin.

9. The apparatus according to claim 7, wherein the non-absorbent surface has a surface tension of more than 40 dynes/cm.

10. The apparatus according to claim 5, wherein the substance delivery area is configured to prevent the substance from spreading across the subject's skin beyond the region of the skin, by generating protrusions in the skin at outer edges of the region, by the substance delivery area applying pressure to the portion of the region of the skin.

11. The apparatus according to claim 10, wherein the substance delivery area has a thickness of 0.5 mm to 1.5 mm.

12. The apparatus according to claim 10, wherein the substance delivery area is configured to apply the pressure to the skin underneath the substance delivery area, by the substance support area being stretched when the substance delivery area is brought into contact with the portion of the region of the skin.

13. The apparatus according to claim 10,
wherein the patch assembly further comprises a first adhesive layer disposed on the substance support area, and
wherein, subsequent to the substance delivery area being brought into contact with the portion of the region of the skin, the first adhesive layer is configured to adhere the substance support area to the frame.

14. The apparatus according to claim 13, wherein the patch assembly further comprises a second adhesive layer disposed on the frame and configured to adhere the frame to the subject's skin.

15. The apparatus according to claim 14, wherein a tackiness of the second adhesive layer is greater than a tackiness of the first adhesive layer.

16. The apparatus according to claim 15, wherein the first adhesive layer is composed of a substance that is the same as a substance of which the second adhesive layer is composed, and wherein the second adhesive layer is thicker than the first adhesive layer.

17. The apparatus according to claim 15, wherein the second adhesive layer comprises a substance that the first adhesive layer does not comprise.

18. A method for delivering a substance through skin of a subject, comprising: placing on the skin a frame of an assembly that surrounds at least in part an open portion of the assembly, such that a region of the skin is exposed through the open portion when the frame is placed on the skin;
bringing a pad into contact, through the open portion, with at least a portion of the region of the skin, the pad having the substance disposed thereon; and
preventing the substance from spreading across the subject's skin beyond the region of the skin, by generating protrusions in the skin at outer edges of the region, by applying pressure, with the pad, to the portion of the region of the subject's skin.

19. The method according to claim 18, wherein applying the pressure with the pad comprises stretching a substance support area on which the pad is disposed, while the pad is brought into contact with the portion of the region of the skin.

20. The method according to claim 18, wherein preventing the substance from spreading across the subject's skin beyond the region of the skin comprises adhering a substance support area to the frame, the pad being disposed on the substance support area.

21. An apparatus for use with skin of a subject, comprising:
a dried substance configured to be delivered through the skin; and
a patch assembly configured to be placed on the skin, the patch assembly comprising:
a frame that surrounds at least in part an open portion of the patch assembly, such that a region of the skin is exposed through the open portion when the frame is placed on the skin; and
a pad having a skin-facing non-absorbent layer, the dried substance being disposed on the skin-facing non-absorbent layer, and the skin-facing non-absorbent layer being configured to:
deliver the dried substance through the skin by being brought into contact, through the open portion, with at least a portion of the region of the skin, the portion of the region of the skin having been wetted, and
prevent resorption of the substance into the pad after the substance has been wetted,
the pad being configured to prevent the substance from spreading across the subject's skin beyond the region of the skin, by generating protrusions in the skin at outer edges of the region, by the pad applying pressure to the portion of the region of the skin.

22. An apparatus for use with skin of a subject, comprising:
a pad having a skin-facing non-absorbent surface; and
a dried substance configured to be delivered through the skin, by being disposed on the non-absorbent surface of the pad and by the non-absorbent surface of the pad being placed on a wetted portion of the skin, the non-absorbent surface being configured to prevent resorption of the substance into the pad after the substance has been wetted.

23. The apparatus according to claim 22, further comprising a skin preparation device comprising one or more electrodes, and configured to wet the substance by facilitating release of fluid from the skin by ablating the subject's skin.

24. The apparatus according to claim 22, wherein the non-absorbent surface has a surface tension of more than 40 dynes/cm.

25. A method for use with skin of a subject, comprising:
providing a pad having a skin-facing non-absorbent surface, a dried form of a substance being disposed on the surface;
causing release of fluid from the skin by ablating the skin; and
delivering the substance through the skin, by wetting the substance, by placing the skin-facing non-absorbent surface of the pad on the ablated skin, the non-absorbent surface preventing resorption of the substance into the pad after the substance has been wetted.

26. Apparatus for facilitating delivery of a substance through skin of a subject, comprising:
a patch assembly configured to deliver the substance through the skin by being placed on the subject's skin, the patch assembly comprising:
a frame that surrounds at least in part an open portion of the patch assembly, such that a region of the skin is exposed through the open portion when the frame is placed on the skin; and
a pad comprising the substance, the pad being adapted to be brought into contact, through the open portion, with at least a portion of the region of the skin, the pad being configured to prevent the substance from spreading across the subject's skin beyond the region of the skin, by generating protrusions in the skin at outer edges of the region, by the pad applying pressure to the portion of the region of the skin.

27. The apparatus according to claim 26, wherein the pad has a thickness of 0.5 mm to 1.5 mm.

28. The apparatus according to claim 26, wherein the patch assembly further comprises a substance support area, wherein the pad is disposed on the substance support area, and wherein the pad is configured to apply the pressure to the skin underneath the pad, by the substance support area being stretched when the pad is brought into contact with the portion of the region of the skin.

29. The apparatus according to claim 28,
wherein the patch assembly further comprises a substance support area and a first adhesive layer,
wherein the pad is disposed on the substance support area, and
wherein, subsequent to the pad being brought into contact with the portion of the region of the skin, the first adhesive layer is configured to adhere the substance support area to the frame.

30. The apparatus according to claim 29, wherein the patch assembly further comprises a second adhesive layer configured to adhere the frame to the subject's skin.

31. The apparatus according to claim 30, wherein a tackiness of the second adhesive layer is greater than a tackiness of the first adhesive layer.

32. The apparatus according to claim 31, wherein the first adhesive layer is composed of a substance that is the same as a substance of which the second adhesive layer is composed, and wherein the second adhesive layer is thicker than the first adhesive layer.

33. The apparatus according to claim 31, wherein the second adhesive layer comprises a substance that the first adhesive layer does not comprise.
